SEQUENCE LISTING (17 sequences)

SEQ ID NO: 1 - protein, 122 aa, Homo sapien
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSS

SEQ ID NO: 2 - protein, 107 aa, Homo sapien
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIK

SEQ ID NO: 3 - protein, 5 aa, Homo sapien
DYAMH

SEQ ID NO: 4 - protein, 17 aa, Homo sapien
TISWNSGSIGYADSVKG

SEQ ID NO: 5 - protein, 13 aa, Homo sapien
DIQYGNYYYGMDV

SEQ ID NO: 6 - protein, 11 aa, Homo sapien
RASQSVSSYLA

SEQ ID NO: 7 - protein, 7 aa, Homo sapien
DASNRAT

SEQ ID NO: 8 - protein, 9 aa, Homo sapien
QQRSNWPIT

SEQ ID NO: 9 - protein, 17 aa, Homo sapien
DYYGAGSFYDGLYGMDV

SEQ ID NO: 10 - protein, 81 aa, Homo sapien
DYAMHWVRQAPGKGLEWVSTISWNSGSIGYADSVKGRFTISRDNAKKSLYLQMNSLRAEDTALYYCAKDIQYGNYYYGMDV

SEQ ID NO: 11 - protein, 14 aa, Homo sapien
DNQYGSGSTYGLGV

SEQ ID NO: 12 - protein, 125 aa, Homo sapien
EVQLVQSGGGLVHPGGSLRLSCAGSGFTFSSYAMHWVRQAPGKGLEWVSAIGTGGGTYYADSVKGRFTISRDNAKNSLYLQMNSLRAEDMAVYYCARDYYGSGSYYYYYYGMDVWGQGTTVTVSS

SEQ ID NO: 13 - protein, 107 aa, Homo sapien
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGGGTKVEIK

SEQ ID NO: 14 - protein, 122 aa, Homo sapien
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDIDYYYYYYGMDVWGQGTTVTVSS

SEQ ID NO: 15 - protein, 107 aa, Homo sapien; identical to SEQ ID NO: 2

SEQ ID NO: 16 - protein, 157 aa, Homo sapien
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 17 - protein, 157 aa, Murine
NFGRLHCTTAVIRNINDQVLFVDKRQPVFEDMTDIDQSASEPQTRLIIYMYKDSEVRGLAVTLSVKDSKMSTLSCKNKIISFEEMDPPENIDDIQSDLIFFQKRVPGHNKMEFESSLYEGHFLACQKEDDAFKLILKKKDENGDKSVMFTLTNLHQS

(19) United States
(12) Patent Application Publication
(10) Pub. No.: US 2008/0274078 A1
(43) Pub. Date: Nov. 6, 2008

(54) NOVEL USES

(76) Inventors: Zdenka HASKOVA, King of Prussia, PA (US); Zdenka Ludmila JONAK, King of Prussia, PA (US); Stephen H. TRULLI, King of Prussia, PA (US); John F. TOSO, King of Prussia, PA (US); Margaret N. WHITACRE, King of Prussia, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 12/052,120

(22) Filed: Mar. 20, 2008

Related U.S. Application Data
(60) Provisional application No. 60/896,855, filed on Mar. 23, 2007.
(60) Provisional application No. 60/952,002, filed on Jul. 26, 2007.

(51) Int. Cl. A61K 38/20; A61P 35/00

(52) U.S. Cl. 424/85.2

(57) ABSTRACT

The present invention relates generally to the use of human IL-18 combinations in the treatment of cancers. In particular, the present invention relates to combination of human IL-18 and an anti-CD20 antibody.

### CROSS REFERENCE TO PRIOR APPLICATIONS

This application claims priority to U.S. provisional applications 60/896,855 filed Mar. 23, 2007 and 60/952,002 filed Jul. 25, 2007.

### FIELD OF INVENTION

The present invention relates generally to the use of IL-18, also known as interferon-γ-inducing factor (IGIF), in combination with a monoclonal antibody that is expressed on the surface of a cancer cell.

### BACKGROUND OF THE INVENTION

Active human IL-18 contains 157 amino acid residues. It has potent biological activities, including induction of interferon-γ-production by T cells and splenocytes, enhancement of the killing activity of NK cells and promotion of the differentiation of naive CD4⁺T cells into Th1 cells. In addition, human IL-18 augments the production of GM-CSF and decreases the production of IL-10. CD4⁺ T cells are the central regulatory elements of all immune responses. They are divided into two subsets, Th1 and Th2. Each subset is defined by its ability to secrete different cytokines. Interestingly, the most potent inducers for the differentiation are cytokines themselves. The development of Th2 cells from naive precursors is induced by IL-4. Prior to the discovery of IL-18, IL-12 was thought of as the principal Th1 inducing cytokine.

Th1 cells secrete IL-2, interferon-γ, and TNF-β. Interferon-γ, the signature Th1 cytokine, acts directly on macrophages to enhance their microbiocidal and phagocytic activities. As a result, the activated macrophages can efficiently destroy intracellular pathogens and tumor cells. The Th2 cells produce IL-4, IL-5, IL-6, IL-10 and IL-13, which act by helping B cells develop into antibody-producing cells. Taken together, Th1 cells are primarily responsible for cell-mediated immunity, while Th2 cells are responsible for humoral immunity.

Based upon a broad spectrum of immunostimulatory properties, IL-18 has been studied in a variety of preclinical tumor models. The anti-tumor activity of IL-18, used as a monotherapy, was observed in tumors that were immunogenic. The most potent anti-tumor effects were observed in the advanced tumor (>100 cm³) model of MOPC-315 plasmacytoma (highly immunogenic tumor). In this model, daily administration of murine IL-18 (5 mg/Kg) for approximately 30 days resulted in a reproducible tumor regression and cure. Rechallenge with parental tumor resulted in tumor rejection, suggesting induction of immunological memory. Additional evidence for involvement of cellular immunity in this model comes from experiments conducted in severe combined immunodeficient mice (SCIDs) bearing advanced MOPC-315 tumors that failed to regress when using a similar schedule of IL-18. Further support for IL-18 mediated cellular immunity also comes from immunohistochemistry performed on established MOPC-315 tumors in control and IL-18 treated mice. This demonstrated increased cellular infiltrates consisting of CD8⁺ T lymphocytes, NK cells, activated macrophages, and dendritic cells in the IL-18 treated animals relative to controls. In vitro, PBMCs or spleen cells from animals treated with IL-18 showed NK and CTL cytotoxicity against the tumor. In addition, it seems that an intact Fas/Fas ligand pathway is beneficial to anti-tumor response.

Rituximab is a chimeric monoclonal antibody that consists of a murine antigen binding site that recognizes the human CD20 antigen fused to the human IgG1 constant region. Rituximab, as a single agent, has significant activity in indolent NHL. In the pivotal single-arm clinical study of 166 patients with relapsed or refractory indolent NHL, the overall response rate was 48% and the complete response (CR) rate was 6%. McLaughlin, et al., J. Clin. Oncol. 16:2825-2833 (1998). In previously untreated patients with indolent NHL, rituximab therapy has an overall response rate of 64 to 73% and CR rate of 15 to 26%. Hainsworth, et al., Blood 95:3052-3056 (2000); Colombat, et al., Blood 97:101-106 (2001). Moreover, multiple randomized Phase III studies have shown that addition of rituximab to conventional chemotherapy improves the survival of patients with NHL. Marcus, et al., Blood 105:1417-1423 (2005); Marcus, et al., Blood 104:3064-3071 (2004); Hiddemann, et al., Blood 106:3725-3732 (2005); Feugier, et al., J. Clin. Oncol. 23:4117-4126 (2005). However, due to higher toxicity with chemotherapy, monotherapy with rituximab is still considered an option in patients with indolent lymphoma.

Research is ongoing to determine ways of enhancing the anti-tumor activity and improve the efficacy of rituximab. Several mechanisms may contribute to the efficacy of rituximab in vivo. Binding of rituximab to CD20 on the surface of lymphoma cells can trigger intracellular signaling pathways leading to apoptosis or programmed cell death. Shan, et al., Blood 91:1644-1652 (1998); Pedersen, et al., Blood 99:1314-1319 (2002). Moreover, rituximab can activate complement species, causing complement-dependent cytolysis. Cragg, et al., Blood 101:1045-1052 (2003); Manches, et al., Blood 101:949-954 (2003). Accumulating evidence suggests that ADCC collaborates with CDC in eliminating tumor cells after administration of rituximab. Manches, et al., supra; Golay, et al., Haematologica 88:1002-1012 (2003); Clynes, et al., Nat. Med. 6:443-446 (2000). ADCC is triggered when the constant (Fc) region of an antibody binds to Fc receptors on the surface of effector cells, such as NK cells or cells of monocyte/macrophage lineage. In a murine model of human B cell lymphoma, the efficacy of rituximab was abrogated in mice lacking activating Fc receptors. In contrast, monoclonal antibody therapy was enhanced in mice lacking inhibitory Fc receptors. Fc receptor-bearing effector cells were critical for the efficacy of rituximab in this model. A major activating Fc receptor in humans is CD16a (FcγRIIIa), which is expressed by NK cells and monocytes. A polymorphism in the human FcγRIIIa gene at position 158 (phenylalanine versus valine) has been shown to correlate with response to rituximab. The 158VV homozygous genotype is associated with stronger IgG binding to and triggering of ADCC by human NK cells in vitro (Koene, et al., Blood 90:1109-1114 (1997); Dall'Ozzo, et al., Cancer Res. 64:4664-4669 (2004)), and is also associated with a higher rate of response after rituximab therapy. Weng, et al., J. Clin. Oncol. 21:3940-3947 (2003); Cartron, et al., Blood 104:2635-2642 (2004). These data support the hypothesis that also NK cell-mediated ADCC is important for the effectiveness of rituximab therapy in patients with lymphoma.

One strategy for improving the efficacy of rituximab is to administer cytokines that can cause the expansion and/or activation of Fc receptor-bearing effector cells, including NK cells and cells of monocyte/macrophage lineage. Phase I clinical trials have shown that rituximab can be safely given in combination with IL-2, IL-112, or GM-CSF to patients with lymphoma. Rossi, et al., Blood 106:2760 (abst 2432) (2005); McLaughlin, et al., Ann. Oncol. 16 (Suppl 5):v68 (abstr 104) (2005); Ansell, et al., Blood 99:67-74 (2002); Eisenbeis, et al., Clin. Cancer Res. 10:6101-6110 (2004); Gluck, et al., Clin. Cancer Res. 10:2253-2264 (2004); Friedberg, et al., Br. J. Haematol. 117:828-834 (2002). Overall objective response rates of 22 to 79% and complete response rates of 5-45% were observed in these studies. In addition, biomarkers such as absolute NK counts and ex vivo ADCC activity correlated with response rates. Most of these studies included predominantly patients with relapsed and refractory disease and with aggressive lymphoma subtypes (DLBCL and mantle cell lymphoma).

### SUMMARY OF THE INVENTION

In one aspect, the present invention relates to a method of treating cancer in a patient in need thereof, comprising the step of: administering, either simultaneously, or sequentially, to the patient: (i) a human IL-18 polypeptide (SEQ ID NO:16) and; (ii) an antibody against CD20 antigen (otherwise called simply as an anti-CD20 antibody) for preventing and/or treating a tumorigenic disease.

In further embodiment, the present invention relates to a method of treating cancer in a patient in need thereof, comprising a staggered administration of (i) a human IL-18 polypeptide (SEQ ID NO:16) and; (ii) an antibody against CD20 antigen (otherwise called simply as an anti-CD20 antibody) for preventing and/or treating a tumorigenic disease (cancer).

In one aspect, the present invention relates to a human IL-18 polypeptide (SEQ ID NO:16) and an anti-CD20 antibody for use in preventing and/or treating a tumorigenic disease (cancer).

In one aspect, the present invention relates to a human IL-18 polypeptide (SEQ ID NO:16) and an anti-CD20 antibody for simultaneous or sequential use (administration) in preventing and/or treating a tumorigenic disease (cancer).

In one aspect, the present invention relates to use of a human IL-18 polypeptide (SEQ ID NO:16) in the manufacture of a medicament for use in combination with an anti-CD20 antibody for preventing and/or treating a tumorigenic disease (cancer).

In one aspect, the present invention relates to use of an anti-CD20 antibody in the manufacture of a medicament for use in combination with a human IL-18 polypeptide (SEQ ID NO:16) for preventing and/or treating a tumorigenic disease (cancer).

In one aspect, the present invention relates to use of a human IL-18 polypeptide (SEQ ID NO:16) and an anti-CD20 antibody in the manufacture of a medicament for preventing and/or treating a tumorigenic disease (cancer).

In one aspect, the present invention relates to a human IL-18 polypeptide (SEQ ID NO:16) for use in combination with an anti-CD20 antibody in preventing and/or treating a tumorigenic disease (cancer).

In one aspect, the present invention relates to an anti-CD20 antibody for use in combination with a human IL-18 polypeptide (SEQ ID NO:16) in preventing and/or treating a tumorigenic disease (cancer). The human IL-18 polypeptide and the anti-CD20 antibody may be administered separately, sequentially and/or simultaneously. Furthermore, the human IL-18 polypeptide and the anti-CD20 antibody may be administered in a staggered manner.

In one embodiment, human IL-18 polypeptide is administered before the anti-CD20 antibody.

In one embodiment, the anti-CD20 antibody is administered before the human IL-18.

In one embodiment of the invention, the anti-CD20 antibody is monoclonal.

In one embodiment, the anti-CD20 antibody has Fc mediated effector function.

In one embodiment, the anti-CD20 antibody has antibody-dependent-cell-mediated cytoxicity (ADCC) effector function.

In one embodiment of the invention, the anti-CD20 antibody is a chimeric, humanized or human monoclonal antibody.

In one embodiment of the invention, the monoclonal antibody against CD20 (anti-CD20 antibody) is a full-length antibody selected from the group consisting of a full-length IgG1 antibody, a full-length IgG2 antibody, a full-length IgG3 antibody, a full-length IgG4 antibody, a full-length IgM antibody, a full-length IgA1 antibody, a full-length IgA2 antibody, a full-length secretory IgA antibody, a full-length IgD antibody, and a full-length IgE antibody, wherein the antibody is glycosylated in a eukaryotic cell.

In one embodiment of the invention, the anti-CD20 antibody is a full-length antibody, such as a full-length IgG1 antibody.

In one embodiment of the invention, the anti-CD20 antibody is an antibody fragment, such as a scFv or a UniBody™ (a monovalent antibody as disclosed in WO 2007/059782). In one embodiment of the invention, the antibody against CD20 is a binding-domain immunoglobulin fusion protein comprising (i) a binding domain polypeptide in the form of a heavy chain variable region of SEQ ID NO:1 or a light chain variable region of SEQ ID NO:2 that is fused to an immunoglobulin hinge region polypeptide, (ii) an immunoglobulin heavy chain CH2 constant region fused to the hinge region, and (iii) an immunoglobulin heavy chain CH3 constant region fused to the CH2 constant region.

In one embodiment of the invention, the antibody against CD20 binds to mutant P172S CD20 (proline at position 172 mutated to serine) with at least the same affinity as to human CD20.

In one embodiment of the invention, the antibody against CD20 binds to an epitope on CD20

(i) which does not comprise or require the amino acid residue proline at position 172;

(ii) which does not comprise or require the amino acid residues alanine at position 170 or proline at position 172;

(iii) which comprises or requires the amino acid residues asparagine at position 163 and asparagine at position 166;

(iv) which does not comprise or require the amino acid residue proline at position 172, but which comprises or requires the amino acid residues asparagine at position 163 and asparagine at position 166; or

(v) which does not comprise or require the amino acid residues alanine at position 170 or proline at position 172, but which comprises or requires the amino acid residues asparagine at position 163 and asparagine at position 166.

In one embodiment of the invention, the antibody against CD20 binds to an epitope in the small first extracellular loop of human CD20.

In one embodiment of the invention, the antibody against CD20 binds to a discontinuous epitope on CD20.

In one embodiment of the invention, the antibody against CD20 binds to a discontinuous epitope on CD20, wherein the epitope comprises part of the first small extracellular loop and part of the second extracellular loop.

In one embodiment of the invention, the antibody against CD20 binds to a discontinuous epitope on CD20, wherein the epitope has residues AGIYAP of the small first extracellular loop and residues MESLNFIRAHTPYI of the second extracellular loop.

In one embodiment of the invention, the antibody against CD20 has one or more of the characteristics selected from the group consisting of:

(i) capable of inducing complement dependent cytotoxicity (CDC) of cells expressing CD20 in the presence of complement;

(ii) capable of inducing complement dependent cytotoxicity (CDC) of cells expressing CD20 and high levels of CD55 and/or CD59 in the presence of complement;

(iii) capable of inducing apoptosis of cells expressing CD20;

(iv) capable of inducing antibody dependent cellular cytotoxicity (ADCC) of cells expressing CD20 in the presence of effector cells;

(v) capable of inducing homotypic adhesion of cells which express CD20;

(vi) capable of translocating into lipid rafts upon binding to CD20;

(vii) capable of depleting cells expressing CD20;

(viii) capable of depleting cells expressing low levels of CD20 (CD20low cells); and

(ix) capable of effectively depleting B cells in situ in human tissues.

In one embodiment of the invention, the antibody against CD20 comprises a VH CDR3 sequence selected from SEQ ID NOs: 5, 9, or 11.

In one embodiment of the invention, the antibody against CD20 comprises a VH CDR1 of SEQ ID NO:3, a VH CDR2 of SEQ ID NO:4, a VH CDR3 of SEQ ID NO:5, a VL CDR1 of SEQ ID NO:6, a VL CDR2 of SEQ ID NO:7 and a VL CDR3 sequence of SEQ ID NO:8.

In one embodiment of the invention, the antibody against CD20 comprises a VH CDR1-CDR3 spanning sequence of SEQ ID NO:10.

In one embodiment of the invention, the antibody against CD20 has human heavy chain and human light chain variable regions comprising the amino acid sequences as set forth in SEQ ID NO:1 and SEQ ID NO:2, respectively; or amino acid sequences which are at least 95% homologous, and more preferably at least 98%, or at least 99% homologous to the amino acid sequences as set forth in SEQ ID NO:1 and SEQ ID NO:2, respectively.

In one embodiment of the invention the CD20 binding molecule is selected from one of the anti-CD20 antibodies disclosed in WO 2004/035607, such as ofatumumab (2F2), 11B8, or 7D8, one of the antibodies disclosed in WO 2005/103081, such as 2C6, one of the antibodies disclosed in WO 2004/103404, AME-133 (humanized and optimized anti-CD20 monoclonal antibody, developed by Applied Molecular Evolution), one of the antibodies disclosed in US 2003/0118592, TRU-015 (CytoxB20G, a small modular immunopharmaceutical fusion protein derived from key domains on an anti-CD20 antibody, developed by Trubion Pharmaceuticals Inc), one of the antibodies disclosed in WO 2003/68821, IMMU-106 (a humanized anti-CD20 monoclonal antibody), one of the antibodies disclosed in WO 2004/56312, ocrelizumab (2H7.v16, PRO-70769, R-1594), Bexxar® (tositumomab), and Rituxan®/MabThera® (rituximab).

The terms “CD20” and “CD20 antigen” are used interchangeably herein, and include any variants, isoforms and species homologs of human CD20, which are naturally expressed by cells or are expressed on cells transfected with the CD20 gene. Synonyms of CD20, as recognized in the art, include B-lymphocyte surface antigen B1, Leu-16 and Bp35. Human CD20 has UniProtKB/Swiss-Prot entry P11836.

The term “immunoglobulin” as used herein refers to a class of structurally related glycoproteins consisting of two pairs of polypeptide chains, one pair of light (L) low molecular weight chains and one pair of heavy (H) chains, all four inter-connected by disulfide bonds. The structure of immunoglobulins has been well characterized. See for instance Fundamental Immunology Ch. 7 (Paul, W., ed., 2nd ed. Raven Press, N.Y. (1989)). Briefly, each heavy chain typically is comprised of a heavy chain variable region (abbreviated herein as VH) and a heavy chain constant region. The heavy chain constant region, CH, typically is comprised of three domains, CH1, CH2, and CH3. Each light chain typically is comprised of a light chain variable region (abbreviated herein as VL) and a light chain constant region. The light chain constant region typically is comprised of one domain, CL. The VH and VL regions may be further subdivided into regions of hypervariability (or hypervariable regions which may be hypervariable in sequence and/or form of structurally defined loops), also termed complementarity determining regions (CDRs), interspersed with regions that are more conserved, termed framework regions (FRs).

Each VH and VL is typically composed of three CDRs and four FRs, arranged from amino-terminus to carboxy-terminus in the following order: FR1, CDR1, FR2, CDR2, FR3, CDR3, FR4 (see also Chothia and Lesk J. Mol. Biol. 196, 901-917 (1987)). Typically, the numbering of amino acid residues in this region is performed by the method described in Kabat et al., Sequences of Proteins of Immunological Interest, 5th Ed. Public Health Service, National Institutes of Health, Bethesda, Md. (1991) (phrases, such as variable domain residue numbering as in Kabat or according to Kabat herein refer to this numbering system for heavy chain variable domains or light chain variable domains). Using this numbering system, the actual linear amino acid sequence of a peptide may contain fewer or additional amino acids corresponding to a shortening of, or insertion into, a FR or CDR of the variable domain. For example, a heavy chain variable domain may include a single amino acid insert (for instance residue 52a according to Kabat) after residue 52 of VH CDR2 and inserted residues (for instance residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

The term “antibody” as used herein refers to an immunoglobulin molecule, a fragment of an immunoglobulin molecule, or a derivative of either thereof, which has the ability to specifically bind to an antigen under typical physiological conditions for a significant period of time, such as at least about 30 minutes, at least about 45 minutes, at least about one hour, at least about two hours, at least about four hours, at least about 8 hours, at least about 12 hours, about 24 hours or more, about 48 hours or more, about 3, 4, 5, 6, 7 or more days, etc., or any other relevant functionally-defined period (such as a time sufficient to induce, promote, enhance, and/or modulate a physiological response associated with antibody binding to the antigen and/or a time sufficient for the antibody to recruit an Fc-mediated effector activity).

The variable regions of the heavy and light chains of the immunoglobulin molecule contain a binding domain that interacts with an antigen. The constant regions of the antibodies may mediate the binding of the immunoglobulin to host tissues or factors, including various cells of the immune system (such as effector cells) and components of the complement system such as C1q, the first component in the classical pathway of complement activation.

The anti-CD20 antibody may be mono-, bi- or multispecific. Indeed, bispecific antibodies, diabodies, and the like, provided by the present invention may bind any suitable target in addition to a portion of CD20.

As indicated above, the term “antibody” as used herein, unless otherwise stated or clearly contradicted by the context, includes fragments of an antibody provided by any known technique, such as enzymatic cleavage, peptide synthesis and recombinant techniques that retain the ability to specifically bind to an antigen. It has been shown that the antigen-binding function of an antibody may be performed by fragments of a full-length (intact) antibody. Examples of antigen-binding fragments encompassed within the term “antibody” include, but are not limited to (i) a Fab fragment, a monovalent fragment consisting of the VL, VH, CL and CH1 domains; (ii) F(ab)₂ and F(ab′)2 fragments, bivalent fragments comprising two Fab fragments linked by a disulfide bridge at the hinge region; (iii) a Fd fragment consisting essentially of the VH and CH1 domains; (iv) a Fv fragment consisting essentially of the VL and VH domains of a single arm of an antibody, (v) a dAb fragment (Ward et al., Nature 341, 544-546 (1989)), which consists essentially of a VH domain and also called domain antibodies (Holt et al. (November 2003) Trends Biotechnol. 21(11):484-90); (vi) a camelid antibody or nanobody (Revets et al. (January 2005) Expert Opin Biol Ther. 5(1):111-24), (vii) an isolated complementarity determining region (CDR), such as a VH CDR3, (viii) a UniBody™, a monovalent antibody as disclosed in WO 2007/059782, (ix) a single chain antibody or single chain Fv (scFv), see for instance Bird et al., Science 242, 423-426 (1988) and Huston et al., PNAS USA 85, 5879-5883 (1988)), (x) a diabody (a scFv dimer), which can be monospecific or bispecific (see for instance PNAS USA 90(14), 6444-6448 (1993), EP 404097 or WO 93/11161 for a description of diabodies), a triabody or a tetrabody. Although such fragments are generally included within the definition of an antibody, they collectively and each independently are unique features of the present invention, exhibiting different biological properties and utility. These and other useful antibody fragments in the context of the present invention are discussed further herein.

It should be understood that the term antibody generally includes monoclonal antibodies as well as polyclonal antibodies. The antibodies can be human, humanized, chimeric, murine, etc. An antibody as generated can possess any isotype.

The term “human antibody”, as used herein, is intended to include antibodies having variable and constant regions derived from human germline immunoglobulin sequences. The human antibodies of the present invention may include amino acid residues not encoded by human germline immunoglobulin sequences (for instance mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted into human framework sequences.

As used herein, a human antibody is “derived from” a particular germline sequence if the antibody is obtained from a system using human immunoglobulin sequences, for instance by immunizing a transgenic mouse carrying human immunoglobulin genes or by screening a human immunoglobulin gene library, and wherein the selected human antibody is at least 90%, such as at least 95%, for instance at least 96%, such as at least 97%, for instance at least 98%, or such as at least 99% identical in amino acid sequence to the amino acid sequence encoded by the germline immunoglobulin gene. Typically, a human antibody derived from a particular human germline sequence will display no more than 10 amino acid differences, such as no more than 5, for instance no more than 4, 3, 2, or 1 amino acid difference from the amino acid sequence encoded by the germline immunoglobulin gene. For VH antibody sequences the VH CDR3 domain is not included in such comparison.

The term “chimeric antibody” refers to an antibody that contains one or more regions from one antibody and one or more regions from one or more other antibodies. The term “chimeric antibody” includes monovalent, divalent, or polyvalent antibodies. A monovalent chimeric antibody is a dimer (HL)) formed by a chimeric H chain associated through disulfide bridges with a chimeric L chain. A divalent chimeric antibody is a tetramer (H2L2) formed by two HL dimers associated through at least one disulfide bridge. A polyvalent chimeric antibody may also be produced, for example, by employing a CH region that assembles into a molecule with 2+ binding sites (for instance from an IgM H chain, or μ chain). Typically, a chimeric antibody refers to an antibody in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see for instance U.S. Pat. No. 4,816,567 and Morrison et al., PNAS USA 81, 6851-6855 (1984)). Chimeric antibodies are produced by recombinant processes well known in the art (see for instance Cabilly et al., PNAS USA 81, 3273-3277 (1984), Morrison et al., PNAS USA 81, 6851-6855 (1984), Boulianne et al., Nature 312, 643-646 (1984), EP125023, Neuberger et al., Nature 314, 268-270 (1985), EP171496, EP173494, WO 86/01533, EP184187, Sahagan et al., J. Immunol. 137, 1066-1074 (1986), WO 87/02671, Liu et al., PNAS USA 84, 3439-3443 (1987), Sun et al., PNAS USA 84, 214-218 (1987), Better et al., Science 240, 1041-1043 (1988) and Harlow et al., Antibodies: A Laboratory Manual, Cold Spring Harbor Laboratory Press, Cold Spring Harbor, N.Y., (1988)).

The term “humanized antibody” refers to a human antibody which contain minimal sequences derived from a non-human antibody. Typically, humanized antibodies are human immunoglobulins (recipient antibody) in which residues from a hypervariable region of the recipient are replaced by residues from a hypervariable region of a non-human species (donor antibody), such as mouse, rat, rabbit or non-human primate having the desired specificity, affinity, and capacity.

Furthermore, humanized antibodies may comprise residues which are not found in the recipient antibody or in the donor antibody. These modifications are made to further refine antibody performance. In general, a humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. A humanized antibody optionally also will comprise at least a portion of a human immunoglobulin constant region. For further details, see Jones et al., Nature 321, 522-525 (1986), Riechmann et al., Nature 332, 323-329 (1988) and Presta, Curr. Op. Struct. Biol. 2, 593-596 (1992).

The terms “monoclonal antibody” or “monoclonal antibody composition” as used herein refer to a preparation of antibody molecules of single molecular composition. A monoclonal antibody composition displays a single binding specificity and affinity for a particular epitope. Accordingly, the term “human monoclonal antibody” refers to antibodies displaying a single binding specificity which have variable and constant regions derived from human germline immunoglobulin sequences. The human monoclonal antibodies may be generated by a hybridoma which includes a B cell obtained from a transgenic or transchromosomal nonhuman animal, such as a transgenic mouse, having a genome comprising a human heavy chain transgene and a light chain transgene, fused to an immortalized cell.

The term “recombinant human antibody”, as used herein, includes all human antibodies that are prepared, expressed, created or isolated by recombinant means, such as (a) antibodies isolated from an animal (such as a mouse) that is transgenic or transchromosomal for human immunoglobulin genes or a hybridoma prepared therefrom (described further elsewhere herein), (b) antibodies isolated from a host cell transformed to express the antibody, such as from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable and constant regions derived from human germline immunoglobulin sequences. In certain embodiments, however, such recombinant human antibodies may be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

The terms “transgenic, non-human animal” refers to a non-human animal having a genome comprising one or more human heavy and/or light chain transgenes or transchromosomes (either integrated or non-integrated into the animal's natural genomic DNA) and which is capable of expressing fully human antibodies. For example, a transgenic mouse can have a human light chain transgene and either a human heavy chain transgene or human heavy chain transchromosome, such that the mouse produces human anti-CD20 antibodies when immunized with CD20 antigen and/or cells expressing CD20. The human heavy chain transgene may be integrated into the chromosomal DNA of the mouse, as is the case for transgenic mice, for instance the HuMAb-Mouse®, such as HCo7 or HCo12 mice, or the human heavy chain transgene may be maintained extrachromosomally, as is the case for the transchromosomal KM-Mouse® as described in WO 02/43478. Such transgenic and transchromosomal mice (collectively referred to herein as “transgenic mice”) are capable of producing multiple isotypes of human monoclonal antibodies to a given antigen (such as IgG, IgA, IgM, IgD and/or IgE) by undergoing V-D-J recombination and isotype switching. Transgenic, nonhuman animals can also be used for production of antibodies against a specific antigen by introducing genes encoding such specific antibody, for example by operatively linking the genes to a gene which is expressed in the milk of the animal.

Tumorigenic diseases (cancers) which can be prevented and/or treated include B cell lymphoma, e.g., NHL (non-Hodgkin's lymphoma), including precursor B cell lymphoblastic leukemia/lymphoma and mature B cell neoplasms, such as B cell chronic lymhocytic leukemia (CLL)/small lymphocytic lymphoma (SLL), B cell prolymphocytic leukemia, lymphoplasmacytic lymphoma, mantle cell lymphoma (MCL), follicular lymphoma (FL), including low-grade, intermediate-grade and high-grade FL, cutaneous follicle center lymphoma, marginal zone B cell lymphoma (MALT type, nodal and splenic type), hairy cell leukemia, diffuse large B cell lymphoma, Burkitt's lymphoma, plasmacytoma, plasma cell myeloma, post-transplant lymphoproliferative disorder, Waldenstrom's macroglobulinemia, anaplastic large-cell lymphoma (ALCL), T-cell Non-Hodgkin's lymphoma; Hodgkin's lymphoma; and melanoma.

### BRIEF DESCRIPTION OF THE FIGURES

FIG. 1 shows the amino acid sequence of native human IL-18 (SEQ ID NO:16).

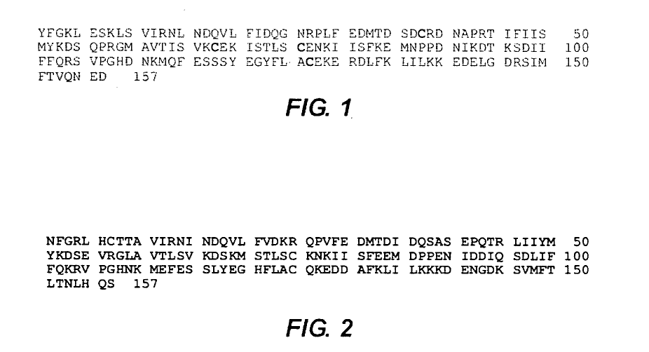

FIG. 2 shows the amino acid sequence of murine IL-18 (SEQ ID NO:17).

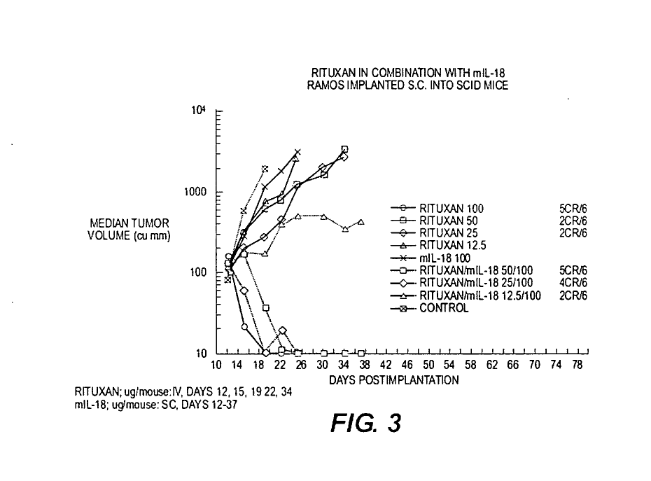

FIG. 3 shows the anti-tumor activity of mIL-18 (SEQ ID NO:17) in combination with Rituxan® in a human B-cell lymphoma murine model. CR stands for complete regression.

FIG. 4 shows the statistical significance when the data from FIG. 3 are graphed and analyzed using GraphPad Prism®. Specifically, this figure compares tumor volumes on day 19 post-implantation.

FIG. 5 shows the tumor volume on day 25 post-implantation of the murine IL-18 (SEQ ID NO:17)/Rituxan® combination in a human B-cell lymphoma model.

FIGS. 6A and 6B shows median and mean tumor growth volume of the murine IL-18 (SEQ ID NO:17)/Rituxan® combination in a human B-cell lymphoma model.

FIGS. 7 and 8 show tumor volume on day 27 post-implantation of the murine IL-18 (SEQ ID NO:17)/Rituxan® combination in a human B-cell lymphoma model, versus either agent alone.

FIG. 9 shows the effect of ofatumumab (HuMax-CD20®) as a monotherapy, or in combination with murine IL-18 (SEQ ID NO:17) on the growth of subcutaneous Ramos human lymphoma in SCID mice (n=6 mice/group; mean & SD). (HuMax refers to ofatumumab.)

FIG. 10 shows the effect of ofatumumab as monotherapy or in combination with murine IL-18 (SEQ ID NO:17) in a s.c. Ramos human lymphomamodel in SCID mice on day 28 after inoculation. (n=6 mice/group; mean +/−SD). (HuMax refers to ofatumumab.)

### DETAILED DESCRIPTION OF THE INVENTION

Since tumors are usually non-immunogenic the focus of pre-clinical studies is focused on combination therapies of IL-18 with monoclonal antibodies. Combining two different agents, each with different mechanism of tumor killing, results in synergistic anti-tumor activity. Examples of IL-18 combination therapies are presented below.

Example 1 focuses on the use of IL-18 in combination with Rituxan® in a human B-cell lymphoma. The aim of this study is to investigate whether the combination of IL-18 and Rituxan® in the human B cell lymphoma model offers a benefit over the monotherapy with IL-18, or Rituxan® alone. The combination of IL-18 with monoclonal antibodies, for example IL-18 with rituximab (Rituxan®) showed synergistic anti-tumor activity in an advanced stage tumor model (SCID mouse xenograft). Since rituximab is only binding to human tumor cells that express CD20, the assessment of anti-tumor activity was performed in the human lymphoma xenograft model in SCID mice.

Another example was investigated whether the combination of IL-18 with other clinically relevant cancer treatments would result in enhanced anti-tumor activity that was superior to monotherapy alone. We have now demonstrated that ofatumumab has synergistic effect with IL-18 when used in combination therapy in Ramos human xenograft model. Thus combination of anti-CD20 antibody with IL-18 offers a benefit over the monotherapy with IL-18 or anti-CD20 antibody alone.

Combination with monoclonal antibodies offers a potential for enhancement of ADCC mechanism of tumor cell killing. Antibodies to CD20 show enhancement of anti-tumor activity in combination with mIL-18 (SEQ ID NO:17). Several mechanisms may contribute to the efficacy of, for example, Rituxan®; however, accumulating evidence suggests that ADCC plays an important role in elimination of tumor cells after administration of Rituxan®. ADCC is triggered when the constant (Fc) region of an antibody binds to Fc receptors on the surface of effector cells, such as natural killer (NK) cells or cells of monocyte/macrophage lineage. A study in mice lacking Fc receptors showed that effect of Rituxan on human B cell lymphoma was abrogated (Uchida et al. 2004; 199 (12): 1659). Thus, Fc receptor-bearing effector cells were critical for the efficacy of Rituxan®. CD16a (FcγRIIIa) is an important Fc receptor in humans, which is expressed by NK cells and macrophages. The data in Example 1 support the hypothesis that NK cell-mediated ADCC is important for the effectiveness of Rituxan® therapy in patients with lymphoma.

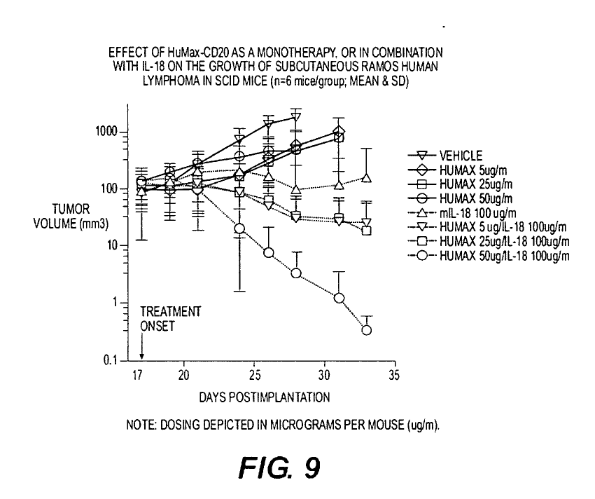

One promising strategy for improving the efficacy of Rituxan® is to administer cytokines, such as IL-18, that can cause the expansion and/or activation of Fc receptor-bearing effector cells, including NK cells and cells of monocyte/macrophage lineage. The pre-clinical mouse tumor model studies with IL-18 in combination with Rituxan® in Example 1 showed benefit over the monotherapies. In this model, the full benefit of IL-18 could not be tested, since the model required human xenograft in the SCID immuno-compromised mouse that has only NK functional cells. The data in Example 1 support that expansion of these ADCC NK effector cells showed benefit in the IL-18 and Rituxan® combo. Rituxan® was active as monotherapy at the highest dose tested. However, similar levels of activity could be seen when lower doses of Rituxan® were used in combination with mIL-18 (SEQ ID NO:17), indicating both that the model was sensitive to the mechanism of Rituxan®, and that the response could be enhanced by IL-18. Moreover, we show here (FIG. 9, 10) that another anti-CD20 antibody (Ofatumumab, HuMax-CD20) has similar effects synergistic with IL-18. Therefore it is believed that combinations of IL-18 with any other anti-CD20 antibodies would show the same synergistic effects.

Example 3 is a Phase I clinical protocol to evaluate the safety and biological activity of IL-18 in combination with rituximab in patients with CD20+ B cell non-Hodgkin's lymphoma (NHL). This study uses a standard treatment regimen of rituximab in combination with rising doses of IL-18 to identify a dose that is safe and tolerable and gives a maximum biological effect, as demonstrated by selected biomarkers (e.g., activated NK cells). Given the good safety and tolerability profile of IL-18 when administered as monotherapy to patients with metastatic melanoma, it is not anticipated that the maximum tolerated dose (MTD) of the combination will be reached in the study; however, this study is designed to define the MTD, if dose-limiting toxicities are identified in patients with non-Hodgkin's lymphoma.

These data in Example 1 shows that the combination of anti-cancer agents with IL-18 has clinical benefit, since these combinations provide two different mechanisms of action: one is a direct effect on the tumor cells, while IL-18 is capable of augmenting a patient's immune cells. These two mechanisms could complement each other, and potentially resulting in long-lasting, superior anti-tumor activity, due to IL-18's capability to generate immunological memory. Overall, Example 1 demonstrates that the combination of IL-18 with antibody to CD20 results in synergy and superior activity.

Human IL-18 polypeptides are disclosed in EP 0692536A2, EP 0712931A2, EP0767178A1, and WO 97/2441. The amino acid sequence of native human IL-18 (“hIL-18”) is set forth in SEQ ID NO:16. Human IL-18 polypeptides are interferon-γ-inducing polypeptides. They play a primary role in the induction of cell-mediated immunity, including induction of interferon-γ production by T cells and splenocytes, enhancement of the killing activity of NK cells, and promotion of the differentiation of naive CD4+ T cells into Th1 cells.

### IL-18 Polypeptides

The IL-18 polypeptides of the present invention can be recovered and purified from recombinant cell cultures by well known methods, including ammonium sulfate or ethanol precipitation, acid extraction, anion or cation exchange chromatography, phosphocellulose chromatography, hydrophobic interaction chromatography, affinity chromatography, hydroxylapatite chromatography, lectin chromatography, and high performance liquid chromatography. Well known techniques for refolding proteins may be employed to regenerate active conformation when the polypeptide is denatured during intracellular synthesis, isolation and/or purification. Methods to purify and produce active human IL-18 are set forth in WO 01/098455.

The present invention also provides pharmaceutical compositions comprising human IL-18 polypeptides (SEQ ID NO:16). Such compositions comprise a therapeutically effective amount of a compound, and may further comprise a pharmaceutically acceptable carrier, diluent, or excipient. Such pharmaceutical carriers can be sterile liquids, such as water and oils, including those of petroleum, animal, vegetable or synthetic origin, such as peanut oil, soybean oil, mineral oil, sesame oil, etc. Water can be used as a carrier when the pharmaceutical composition is administered intravenously. Saline solutions and aqueous dextrose and glycerol solutions can also be employed as liquid carriers, for example, for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations, and the like. The composition can be formulated as a suppository, with traditional binders and carriers, such as triglycerides. Oral formulation can include standard carriers, such as pharmaceutical grades of mannitol, lactose, starch, magnesium stearate, sodium saccharine, cellulose, magnesium carbonate, etc. Examples of suitable pharmaceutical carriers are described in REMINGTON'S PHARMACEUTICAL SCIENCES by E. W. Martin. Such compositions will contain a therapeutically effective amount of the compound, often in purified form, together with a suitable amount of carrier so as to provide the form for proper administration to the patient. The formulation should suit the mode of administration.

In this application, human or murine IL-18 (SEQ ID NO:16 or 17, respectively) are specifically exemplified. However, the spirit of the invention is not limited to specific human and murine IL-18. Thus any polypeptide which has at least 80%, 85%, 90%, 95%, or 99% identity to the amino acid sequence of SEQ ID NO:16 or SEQ 17 can be substituted for either SEQ ID NO: 16 or SEQ ID NO: 17. Thus in a more embodiment, any polypeptide which has at least 80%, 85%, 90%, 95%, or 99% identity to the amino acid sequence of SEQ ID NO:16 or SEQ 17 are defined as IL-18 (or IL-18 polypeptide).

For amino acid (polypeptide) sequences, the term “identity” indicates the degree of identity between two amino acid sequences when optimally aligned and compared with appropriate insertions or deletions.

The percent identity between two sequences is a function of the number of identical positions shared by the sequences (i.e., % identity=# of identical positions/total # of positions times 100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two amino acid sequences can be determined using the algorithm of E. Meyers and W. Miller (Comput. Appl. Biosci., 4:11-17 (1988)) which has been incorporated into the ALIGN program (version 2.0), using a PAM 120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

In one embodiment of the invention, the composition is formulated in accordance with routine procedures as a pharmaceutical composition adapted for intravenous administration to human beings. Typically, compositions for intravenous administration are solutions in sterile isotonic aqueous buffer. Where suitable, the composition may also include a solubilizing agent and a local anesthetic, such as lignocaine, to ease pain at the site of the injection. Generally, the ingredients are supplied either separately or mixed together in unit dosage form, for example, as a dry lyophilized powder, or water-free concentrate, in a hermetically sealed container, such as an ampoule or sachette, indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

Accordingly, the polypeptide may be used in the manufacture of a medicament. Pharmaceutical compositions of the invention may be formulated as solutions or as lyophilized powders for parenteral administration. Powders may be reconstituted by addition of a suitable diluent or other pharmaceutically acceptable carrier prior to use. The liquid formulation may be a buffered, isotonic, aqueous solution. Examples of suitable diluents are normal isotonic saline solution, standard 5% dextrose in water or buffered sodium or ammonium acetate solution. Such a formulation is especially suitable for parenteral administration, but may also be used for oral administration or contained in a metered dose inhaler or nebulizer for insufflation. It may be desirable to add excipients, such as polyvinylpyrrolidone, gelatin, hydroxy cellulose, acacia, polyethylene glycol, mannitol, sodium chloride, or sodium citrate, to such pharmaceutical compositions.

Alternately, the polypeptide may be encapsulated, tableted or prepared in an emulsion or syrup for oral administration. Pharmaceutically acceptable solid or liquid carriers may be added to enhance or stabilize the composition, or to facilitate preparation of the composition. Solid carriers include starch, lactose, calcium sulfate dihydrate, terra alba, magnesium stearate or stearic acid, talc, pectin, acacia, agar, or gelatin. Liquid carriers include syrup, peanut oil, olive oil, saline, and water. The carrier may also include a sustained release material, such as glyceryl monostearate or glyceryl distearate, alone or with a wax. The amount of solid carrier varies but, will be between about 20 mg to about 1 g per dosage unit. The pharmaceutical preparations are made following the conventional techniques of pharmacy involving milling, mixing, granulating, and compressing, when suitable, for tablet forms; or milling, mixing and filling for hard gelatin capsule forms. When a liquid carrier is used, the preparation will be in the form of a syrup, elixir, emulsion, or an aqueous, or non-aqueous suspension. Such a liquid formulation may be administered directly by mouth (p.o.) or filled into a soft gelatin capsule.

Human IL-18 polypeptides may be prepared as pharmaceutical compositions containing an effective amount the polypeptide as an active ingredient in a pharmaceutically acceptable carrier. In the compositions of the invention, an aqueous suspension or solution containing the polypeptide, buffered at physiological pH, in a form ready for injection may be employed. The compositions for parenteral administration will commonly comprise a solution of the polypeptide of the invention or a cocktail thereof dissolved in a pharmaceutically acceptable carrier, such as an aqueous carrier. A variety of aqueous carriers may be employed, e.g., 0.4% saline, 0.3% glycine, and the like. These solutions are sterile and generally free of particulate matter. These solutions may be sterilized by conventional, well known sterilization techniques (e.g., filtration). The compositions may contain pharmaceutically acceptable auxiliary substances as required to approximate physiological conditions such as pH adjusting and buffering agents, etc. The concentration of the polypeptide of the invention in such pharmaceutical formulation can vary widely, i.e., from less than about 0.5%, usually at or at least about 1% to as much as 15 or 20% by weight and will be selected primarily based on fluid volumes, viscosities, etc., according to the particular mode of administration selected.

Thus, a pharmaceutical composition of the invention for intramuscular injection could be prepared to contain 1 mL sterile buffered water, and between about 1 ng to about 100 mg, e.g., about 50 ng to about 30 mg, or from about 5 mg to about 25 mg, of a polypeptide of the invention. Similarly, a pharmaceutical composition of the invention for intravenous infusion could be made up to contain about 250 mL of sterile Ringer's solution, and about 1 mg to about 30 mg, or from about 5 mg to about 25 mg of a polypeptide of the invention. Actual methods for preparing parenterally administrable compositions are well known or will be apparent to those skilled in the art and are described in more detail in, for example, REMINGTON'S PHARMACEUTICAL SCIENCE, 15th ed., Mack Publishing Company, Easton, Pa.

The polypeptides of the invention, when prepared in a pharmaceutical preparation, may be present in unit dose forms. The appropriate therapeutically effective dose can be determined readily by those of skill in the art. Such a dose may, if suitable, be repeated at appropriate time intervals selected as appropriate by a physician during the response period. In addition, in vitro assays may optionally be employed to help identify optimal dosage ranges. The precise dose to be employed in the formulation will also depend upon the route of administration, and the seriousness of the disease or disorder, and should be decided according to the judgment of the practitioner and each patient's circumstances. Effective doses may be extrapolated from dose-response curves derived from in vitro or animal model test systems.

For polypeptides, the dosage administered to a patient is typically 0.1 mg/kg to 100 mg/kg of the patient's body weight. The dosage administered to a patient may be between 0.1 mg/kg and 20 mg/kg of the patient's body weight, or alternatively, 1 mg/kg to 10 mg/kg of the patient's body weight. Generally, human polypeptides have a longer half-life within the human body than polypeptides from other species, due to the immune response to the foreign polypeptides. Thus, lower dosages of human polypeptides and less frequent administration is often possible. Further, the dosage and frequency of administration of polypeptides of the invention may be reduced by enhancing uptake and tissue penetration (e.g., into the brain) of the polypeptides by modifications such as, for example, lipidation.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration. In another embodiment of the invention, a kit can be provided with the appropriate number of containers required to fulfill the dosage requirements for treatment of a particular indication.

In another embodiment, the compound or composition can be delivered in a vesicle, in particular a liposome (see Langer, Science 249:1527-1533 (1990); Treat, et al., in LIPOSOMES IN THE THERAPY OF INFECTIOUS DISEASE AND CANCER, Lopez-Berestein and Fidler (eds.), Liss, New York, pp. 353-365 (1989); Lopez-Berestein, ibid., pp. 317-327; see generally ibid.).

In yet another embodiment, the compound or composition can be delivered in a controlled release system. In one embodiment, a pump may be used (see Langer, supra; Sefton, CRC Crit. Ref. Biomed. Eng. 14:201 (1987); Buchwald, et al., Surgery 88:507 (1980); Saudek, et al., N. Engl. J. Med. 321:574 (1989)). In another embodiment, polymeric materials can be used (see MEDICAL APPLICATIONS OF CONTROLLED RELEASE, Langer and Wise (eds.), CRC Pres., Boca Raton, Fla. (1974); CONTROLLED DRUG BIOAVAILABILITY, DRUG PRODUCT DESIGN AND PERFORMANCE, Smolen and Ball (eds.), Wiley, New York (1984); Ranger, et al., J., Macromol. Sci. Rev. Macromol. Chem. 23:61 (1983); see also Levy, et al., Science 228:190 (1985); During, et al., Ann. Neurol. 25:351 (1989); Howard, et al., J. Neurosurg. 71:105 (1989)). In yet another embodiment, a controlled release system can be placed in proximity of the therapeutic target, i.e., the brain, thus requiring only a fraction of the systemic dose (see, e.g., Goodson, in MEDICAL APPLICATIONS OF CONTROLLED RELEASE, supra, vol. 2, pp. 115-138 (1984)). Other controlled release systems are discussed in the review by Langer (Science 249:1527-1533 (1990)).

Human IL-18 polypeptide (SEQ ID NO:16) may be administered by any appropriate internal route, and may be repeated as needed, e.g., as frequently as one to three times daily for between 1 day to about three weeks to once per week or once biweekly. Alternatively, the peptide may be altered to reduce charge density and thus allow oral bioavailability. The dose and duration of treatment relates to the relative duration of the molecules of the present invention in the human circulation, and can be adjusted by one of skill in the art, depending upon the condition being treated and the general health of the patient.

The invention provides methods of treatment, inhibition and prophylaxis by administration to a human patient an effective amount of a compound or pharmaceutical composition of the invention comprising human IL-18 polypeptide (SEQ ID NO:16). In one embodiment of the invention, the compound is substantially purified (e.g., substantially free from substances that limit its effect or produce undesired side-effects). Formulations and methods of administration can be employed when the compound comprises a polypeptide as described above; additional appropriate formulations and routes of administration can be selected from among those described herein below.

Various delivery systems are known and can be used to administer a compound of the invention, e.g., encapsulation in liposomes, microparticles, microcapsules, recombinant cells capable of expressing the compound, receptor-mediated endocytosis (see, e.g., Wu, et al., J. Biol. Chem. 262:4429-4432 (1987)), construction of a nucleic acid as part of a retroviral or other vector, etc. Methods of introduction include, but are not limited to, intradermal, intramuscular, intraperitoneal, intravenous, subcutaneous, intranasal, epidural, and oral routes. The compounds or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compounds or compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

### Anti-CD20 Antibodies

A physician or veterinarian having ordinary skill in the art can readily determine and prescribe the effective amount of the pharmaceutical composition comprising anti-CD20 antibody. For example, the physician or veterinarian could start doses of the compounds of the invention employed in the pharmaceutical composition at levels lower than that required in order to achieve the desired therapeutic effect and gradually increase the dosage until the desired effect is achieved. In general, a suitable daily dose of a composition of the invention will be that amount of the compound which is the lowest dose effective to produce a therapeutic effect. It is preferred that administration be intravenous, intramuscular, intraperitoneal, or subcutaneous. If desired, the effective daily dose of a therapeutic composition may be administered as two, three, four, five, six or more sub-doses administered separately at appropriate intervals throughout the day, optionally, in unit dosage forms. While it is possible for anti-CD20 antibody to be administered alone, it is preferable to administer the compound as a pharmaceutical formulation (composition).

In one embodiment, the human monoclonal antibodies according to the invention may be administered by infusion in a weekly dosage of 10 to 2000 mg/m², normally 10 to 500 mg/m², such as 200 to 400 mg/m², such as 375 mg/m². Such administration may be repeated, e.g., 1 to 8 times, such as 3 to 5 times. The administration may be performed by continuous infusion over a period of from 2 to 24 hours, such as of from 2 to 12 hours.

In another embodiment, the antibodies are administered by slow continuous infusion over a long period, such as more than 24 hours, in order to reduce toxic side effects.

In still another embodiment the antibodies are administered in a weekly dosage of from 250 mg to 2000 mg, such as for example 300 mg, 500 mg, 700 mg, 1000 mg, 1500 mg or 2000 mg, for up to 8 times, such as from 4 to 6 times. The administration may be performed by continuous infusion over a period of from 2 to 24 hours, such as of from 2 to 12 hours. Such regimen may be repeated one or more times as necessary, for example, after 6 months or 12 months. The dosage can be determined or adjusted by measuring the amount of circulating anti-CD20 antibodies upon administration in a biological sample by using anti-idiotypic antibodies which target the anti-CD20 antibodies.

In yet another embodiment, the antibodies are administered by maintenance therapy, such as, e.g., once a week for a period of 6 months or more.

In one embodiment, the present invention provides a pharmaceutical composition comprising a therapeutically effective amount of an anti-CD20 antibody. The pharmaceutical compositions may be formulated with pharmaceutically acceptable carriers or diluents as well as any other known adjuvants and excipients in accordance with conventional techniques, such as those disclosed in Remington: The Science and Practice of Pharmacy, 19th Edition, Gennaro, Ed., Mack Publishing Co., Easton, Pa., 1995.

A pharmaceutical composition of the present invention may include diluents, fillers, salts, buffers, detergents (e.g., a nonionic detergent, such as Tween-80), stabilizers, stabilizers (e.g., sugars or protein-free amino acids), preservatives, tissue fixatives, solubilizers, and/or other materials suitable for inclusion in a pharmaceutical composition.

The actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

An anti-CD20 antibody of the present invention may be administered via any suitable route, such as an oral, nasal, inhalable, intrabronchial, intraalveolar, topical (including buccal, transdermal and sublingual), rectal, vaginal and/or parenteral route

In one embodiment, a pharmaceutical composition of the present invention is administered parenterally.

The phrases “parenteral administration” and “administered parenterally” as used herein means modes of administration other than enteral and topical administration, usually by injection, and include epidermal, intravenous, intramuscular, intraarterial, intrathecal, intracapsular, intraorbital, intracardiac, intradermal, intraperitoneal, intratendinous, transtracheal, subcutaneous, subcuticular, intraarticular, subcapsular, subarachnoid, intraspinal, intracranial, intrathoracic, epidural and intrasternal injection and infusion.

In one embodiment the pharmaceutical composition is administered by intravenous or subcutaneous injection or infusion. For example the pharmaceutical composition may be administered over 2-8 hours, such as 4 hours, in order to reduce side effects.

In one embodiment the pharmaceutical composition is administered by inhalation. Fab fragments of an anti-CD20 antibodies may be suitable for such administration route, cf. Crowe et al. (Feb. 15, 1994) Proc Natl Acad Sci USA, 91(4):1386-1390.

In one embodiment the pharmaceutical composition is administered in crystalline form by subcutaneous injection, cf. Yang et al., PNAS USA 100(12), 6934-6939 (2003). Regardless of the route of administration selected, an anti-CD20 antibody, which may be used in the form of a pharmaceutically acceptable salt or in a suitable hydrated form, are formulated into pharmaceutically acceptable dosage forms by conventional methods known to those of skill in the art. A “pharmaceutically acceptable salt” refers to a salt that retains the desired biological activity of the parent compound and does not impart any undesired toxicological effects (see for instance Berge, S. M. et al., J. Pharm. Sci. 66, 1-19 (1977)). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous acids and the like, as well as from nontoxic organic acids, such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

Pharmaceutically acceptable carriers include any and all suitable solvents, dispersion media, coatings, antibacterial and antifungal agents, isotonicity agents, antioxidants and absorption delaying agents, and the like that are physiologically compatible with a compound of the present invention.

Examples of suitable aqueous and nonaqueous carriers which may be employed in the pharmaceutical compositions of the present invention include water, saline, phosphate buffered saline, ethanol, dextrose, polyols (such as glycerol, propylene glycol, polyethylene glycol, and the like), and suitable mixtures thereof, vegetable oils, such as olive oil, corn oil, peanut oil, cottonseed oil, and sesame oil, carboxymethyl cellulose colloidal solutions, tragacanth gum and injectable organic esters, such as ethyl oleate, and/or various buffers. Other carriers are well known in the pharmaceutical arts.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the present invention is contemplated.

Proper fluidity may be maintained, for example, by the use of coating materials, such as lecithin, by the maintenance of the required particle size in the case of dispersions, and by the use of surfactants.

Pharmaceutical compositions containing an anti-CD20 antibody may also comprise pharmaceutically acceptable antioxidants for instance (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Pharmaceutical compositions of the present invention may also comprise isotonicity agents, such as sugars, polyalcohols such as mannitol, sorbitol, glycerol or sodium chloride in the compositions.

Pharmaceutically acceptable diluents include saline and aqueous buffer solutions.

The pharmaceutical compositions containing an anti-CD20 antibody may also contain one or more adjuvants appropriate for the chosen route of administration, such as preservatives, wetting agents, emulsifying agents, dispersing agents, preservatives or buffers, which may enhance the shelf life or effectiveness of the pharmaceutical composition. An anti-CD20 antibody the present invention may for instance be admixed with lactose, sucrose, powders (e.g., starch powder), cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, and/or polyvinyl alcohol. Other examples of adjuvants are QS21, GM-CSF, SRL-172, histamine dihydrochloride, thymocartin, Tio-TEPA, monophosphoryl-lipid A/microbacteria compositions, alum, incomplete Freund's adjuvant, montanide ISA, ribi adjuvant system, TiterMax adjuvant, syntex adjuvant formulations, immune-stimulating complexes (ISCOMs), gerbu adjuvant, CpG oligodeoxynucleotides, lipopolysaccharide, and polyinosinic:polycytidylic acid.

Prevention of presence of microorganisms may be ensured both by sterilization procedures and by the inclusion of various antibacterial and antifungal agents, for example, paraben, chlorobutanol, phenol, sorbic acid, and the like. In addition, prolonged absorption of the injectable pharmaceutical form may be brought about by the inclusion of agents which delay absorption, such as aluminum monostearate and gelatin.

The pharmaceutical compositions containing an anti-CD20 antibody may be in a variety of suitable forms. Such forms include, for example, liquid, semi-solid and solid dosage forms, such as liquid solutions (e.g., injectable and infusible solutions), dispersions or suspensions, emulsions, microemulsions, gels, creams, granules, powders, tablets, pills, powders, liposomes, dendrimers and other nanoparticles (see for instance Baek et al., Methods Enzymol. 362, 240-9 (2003), Nigavekar et al., Pharm Res. 21(3), 476-83 (2004), microparticles, and suppositories.

The optimal form depends on the mode of administration chosen and the nature of the composition. Formulations may include, for instance, powders, pastes, ointments, jellies, waxes, oils, lipids, lipid (cationic or anionic) containing vesicles, DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing may be appropriate in treatments and therapies in accordance with the present invention, provided that the anti-CD20 antibody in the pharmaceutical composition is not inactivated by the formulation and the formulation is physiologically compatible and tolerable with the route of administration. See also for instance Powell et al., “Compendium of excipients for parenteral formulations” PDA J Pharm Sci Technol. 52, 238-311 (1998) and the citations therein for additional information related to excipients and carriers well known to pharmaceutical chemists.

An anti-CD20 antibody may be prepared with carriers that will protect the compound against rapid release, such as a controlled release formulation, including implants, transdermal patches, and microencapsulated delivery systems. Such carriers may include gelatin, glyceryl monostearate, glyceryl distearate, biodegradable, biocompatible polymers, such as ethylene vinyl acetate, polyanhydrides, polyglycolic acid, collagen, polyorthoesters, and polylactic acid alone or with a wax, or other materials well known in the art. Methods for the preparation of such formulations are generally known to those skilled in the art. See e.g., Sustained and Controlled Release Drug Delivery Systems, J. R. Robinson, ed., Marcel Dekker, Inc., New York, 1978.

To administer the pharmaceutical compositions containing an anti-CD20 antibody by certain routes of administration according to the invention, it may be necessary to coat the anti-CD20 antibody with, or co-administer the antibody with, a material to prevent its inactivation. For example, the anti-CD20 antibody may be administered to a subject in an appropriate carrier, for example, liposomes, or a diluent. Liposomes include water-in-oil-in-water CGF emulsions as well as conventional liposomes (Strejan et al., J. Neuroimmunol. 7, 27 (1984)).

Depending on the route of administration, an anti-CD20 antibody may be coated in a material to protect the antibody from the action of acids and other natural conditions that may inactivate the compound. For example, the anti-CD20 antibody may be administered to a subject in an appropriate carrier, for example, liposomes. Liposomes include water-in-oil-in-water CGF emulsions as well as conventional liposomes (Strejan et al., J. Neuroimmunol. 7, 27 (1984)).

Pharmaceutically acceptable carriers for parenteral administration include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the present invention is contemplated. Supplementary active compounds may also be incorporated into the compositions.

Pharmaceutical compositions for injection must typically be sterile and stable under the conditions of manufacture and storage. The composition may be formulated as a solution, microemulsion, liposome, or other ordered structure suitable to high drug concentration. The carrier may be a aqueous or nonaqueous solvent or dispersion medium containing for instance water, ethanol, polyols (such as glycerol, propylene glycol, polyethylene glycol, and the like), and suitable mixtures thereof, vegetable oils, such as olive oil, and injectable organic esters, such as ethyl oleate. The proper fluidity may be maintained, for example, by the use of a coating, such as lecithin, by the maintenance of the required particle size in the case of dispersion and by the use of surfactants. In many cases, it will be preferable to include isotonic agents, for example, sugars, polyalcohols, such as glycerol, mannitol, sorbitol, or sodium chloride in the composition. Prolonged absorption of the injectable compositions may be brought about by including in the composition an agent that delays absorption, for example, monostearate salts and gelatin. Sterile injectable solutions may be prepared by incorporating the active compound in the required amount in an appropriate solvent with one or a combination of ingredients e.g. as enumerated above, as required, followed by sterilization microfiltration.

Generally, dispersions are prepared by incorporating the active compound into a sterile vehicle that contains a basic dispersion medium and the required other ingredients e.g. from those enumerated above. In the case of sterile powders for the preparation of sterile injectable solutions, examples of methods of preparation are vacuum drying and freeze-drying (lyophilization) that yield a powder of the active ingredient plus any additional desired ingredient from a previously sterile-filtered solution thereof.

Sterile injectable solutions may be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, examples of methods of preparation are vacuum drying and freeze-drying (lyophilization) that yield a powder of the active ingredient plus any additional desired ingredient from a previously sterile-filtered solution thereof.

The present invention may be embodied in other specific forms, without departing from the spirit or essential attributes thereof, and, accordingly, reference should be made to the appended claims, rather than to the foregoing specification or following examples, as indicating the scope of the invention.

### GLOSSARY

The following definitions are provided to facilitate understanding of certain terms used frequently hereinbefore.

“Antibody-Dependent Cell-Mediated Cytotoxicity (ADCC)” and “Antibody-Dependent Cell-Mediated Cytotoxicity (ADCC) effector function”, as used herein, both pertain to a mechanism of cell-mediated immunity, whereby an effector cell of the immune system actively lyses a target cell that has been bound by specific antibodies. ADCC is one of the mechanisms through which antibodies, as part of the humoral immune response, can act to limit and contain infection. Classical ADCC is mediated by natural killer (NK) cells, but an alternate ADCC is used by eosinophils to kill certain parasitic worms known as helminths. ADCC is part of the adaptive immune response due to its dependence on a prior antibody response.

The typical ADCC involves activation of NK cells and is dependent upon the recognition of antibody-coated infected cells by Fc receptors on the surface of the NK cell. The Fc receptors recognize the Fc (constant) portion of antibodies such as IgG, which bind to the surface of a pathogen-infected target cell. The Fc receptor that exists on the surface of NK Cell is called CD16a or FcγRIIIa. Once bound to the Fc receptor of IgG the Natural Killer cell releases cytokines such as IFN-γ, and cytotoxic granules, such as perform and granzyme, that enter the target cell and promote cell death by triggering apoptosis. This ADCC effector function is similar to, but independent of, responses by cytotoxic T cells (CTLs).

As used herein, the term, “carrier”, refers to a diluent, adjuvant, excipient, or vehicle with which the therapeutic is administered.

“Isolated” means altered “by the hand of man” from its natural state, i.e., if it occurs in nature, it has been changed or removed from its original environment, or both. For example, a polynucleotide or a polypeptide naturally present in a living organism is not “isolated,” but the same polynucleotide or polypeptide separated from at least one of its coexisting cellular materials of its natural state is “isolated”, as the term is employed herein. Moreover, a polynucleotide or polypeptide that is introduced into an organism by transformation, genetic manipulation or by any other recombinant method is “isolated” even if it is still present in said organism, which organism may be living or non-living.

As used herein, the term, “pharmaceutical”, includes veterinary applications of the invention. The term, “therapeutically effective amount”, refers to that amount of therapeutic agent, which is useful for alleviating a selected condition.

As used herein, the term, “pharmaceutically acceptable”, means approved by a regulatory agency of the Federal or a state government or listed in the U.S. Pharmacopeia or other generally recognized pharmacopeia for use in animals, and more particularly in humans.

“Polypeptide” refers to any polypeptide comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the 20 gene-encoded amino acids. “Polypeptides” include amino acid sequences modified either by natural processes, such as post-translational processing, or by chemical modification techniques that are well known in the art. Such modifications are well described in basic texts and in more detailed monographs, as well as in a voluminous research literature. Modifications may occur anywhere in a polypeptide, including the peptide backbone, the amino acid side-chains and the amino or carboxyl termini. It will be appreciated that the same type of modification may be present to the same or varying degrees at several sites in a given polypeptide. Also, a given polypeptide may contain many types of modifications. Polypeptides may be branched as a result of ubiquitination, and they may be cyclic, with or without branching. Cyclic, branched and branched cyclic polypeptides may result from post-translation natural processes or may be made by synthetic methods. Modifications include acetylation, acylation, ADP-ribosylation, amidation, biotinylation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins, such as arginylation, and ubiquitination (see, for instance, PROTEINS—STRUCTURE AND MOLECULAR PROPERTIES, 2nd Ed., T. E. Creighton, W.H. Freeman and Company, New York, 1993; Wold, F., Post-translational Protein Modifications: Perspectives and Prospects, 1-12, in POST-TRANSLATIONAL COVALENT MODIFICATION OF PROTEINS, B. C. Johnson, Ed., Academic Press, New York, 1983; Seifter et al., Meth Enzymol, 182, 626-646, 1990; Rattan, et al., Ann. NY Acad. Sci., 663: 48-62 (1992)).

### BIOLOGICAL METHODS/EXAMPLES

### Example 1

### Experimental Protocol for IL-18 Combination Therapy with Rituxan® in a Murine Human B-Cell Lymphoma Model

Human IL-18 (SEQ ID NO:16) is a recombinant mature form of human interleukin-18, expressed in a non-pathogenic strain of Escherichia coli. IL-18 is a non-glycosylated monomer of 18 Kd with a primary structure most closely related to IL-10 of the IL-1 trefoil sub-family. Murine and human IL-18 cDNA encode a precursor protein consisting of 192 and 193 amino acids (SEQ ID NOs: 17 and 16, respectively). Pro-IL-18 requires processing by caspases into bioactive mature protein (157 amino acids) in order to mediate its biological activity. The homology between human and murine IL-18 is 65%. In the pre-clinical studies outlined below, murine IL-18 (SEQ ID NO:17) was used, in order to provide an in vivo syngeneic system, where the full immunological potential of IL-18 could be analyzed.

The study was performed in outbred female homozygous SCID mice (ICR-Prkdc^{scid}) that lack both T and B cells. The advantage of using the outbred stock over the inbred strain is that the outbred ICR SCID strain does not exhibit leakiness (even in 10-12 month old mice).

Mice were injected with human Ramos B-cell lymphoma line that was originally derived from a 3-year-old patient with Burkitt's lymphoma (ATCC catalogue, CRL 1596). The tumor 1:10 homogenate was inoculated into 6-8 week old mice at the dose 0.5 ml per mouse. The tumor volume was measured 2-3 times a week, and mice were randomly distributed into the treatment groups so that the groups had equal distribution of tumor volumes. The therapy was initiated when the median tumor volume per group reached 80-150 mm³ (at day 12 post tumor inoculation). In addition, those mice that grew a tumor with a volume outside of the set limits were excluded from the study.

In the first study, the treatment groups (n=6) included a control group (no therapy), three Rituxan® I.V. monotherapy groups (12.5, 25, and 50 μg/mouse BIW, respectively), a murine IL-18 S.C. monotherapy group (100 μg/mouse q.d.), and three combinational therapy groups that each received 100 μg/mouse IL-18 S.C. q.d. plus 12.5, 25, or 50 μg/mouse Rituxan® I.V., respectively.

In the second study, the dosing consisted of mIL-18 (SEQ ID NO:17) at 100 μg/mouse on an SID schedule, and Rituxan® at 25 and 12.5 μg on qd4/3 schedule. The number of animals was increased to n=12, in order to have a better window to measure statistical significance. Tumor volume was measured using the viener calipers two to three times a week.

The combinational therapy with IL-18 and Rituxan® in the human B-cell lymphoma model offers a benefit over the monotherapy with either IL-18, or Rituxan® alone. Two experiments, detailed below, show a statistically significant benefit of the combination therapy in this model.

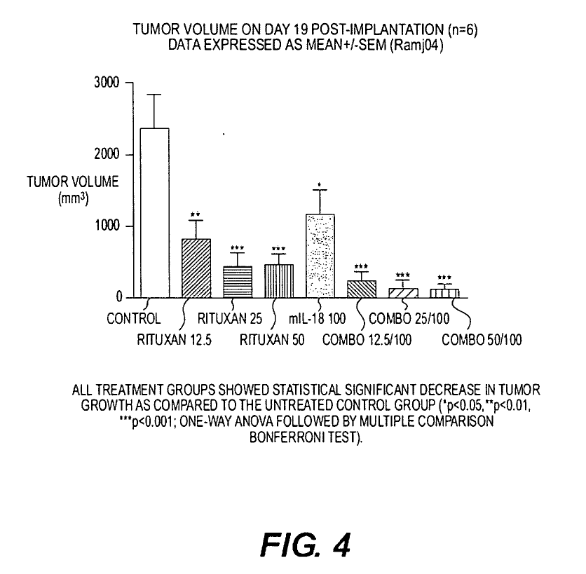

In the first experiment, captured in FIG. 3, the high dose of Rituxan® (100 μg/dose) showed strong anti-tumor activity as a single agent therapy, while at lower dose (12.5 g/dose), Rituxan® had no activity. Murine IL-18 (SEQ ID NO:17) had no activity as a single agent (100 μg/dose). However, when combined with a lower dose of Rituxan®, mIL-18 (SEQ ID NO:17) additive/synergistic activity was shown (12.5 g/dose of Rituxan® combined with 100 μg of mIL-18 (SEQ ID NO:17).

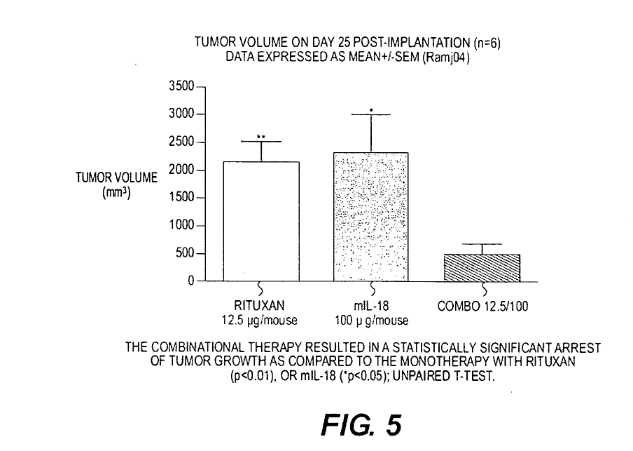

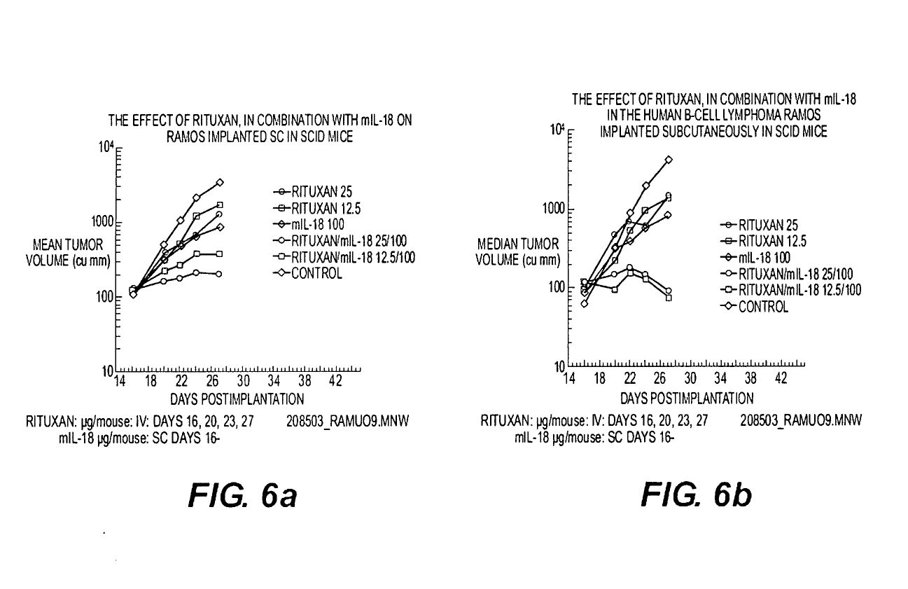

The statistical significance is demonstrated below in FIGS. 4 and 5, when the data are graphed and analysed using GraphPad Prism®. In the first of these graphs, FIG. 4, the tumor volumes are compared on day 19 post-implantation. The statistical analysis showed a significant decrease of tumor growth in all treatment groups as compared to the untreated control group (*p<0.05, **p<0.01, ***p<0.001). The second graph, FIG. 5, shows that the combination therapy was more effective (statistically significant, *p<0.05, **p<0.01) than monotherapies alone.

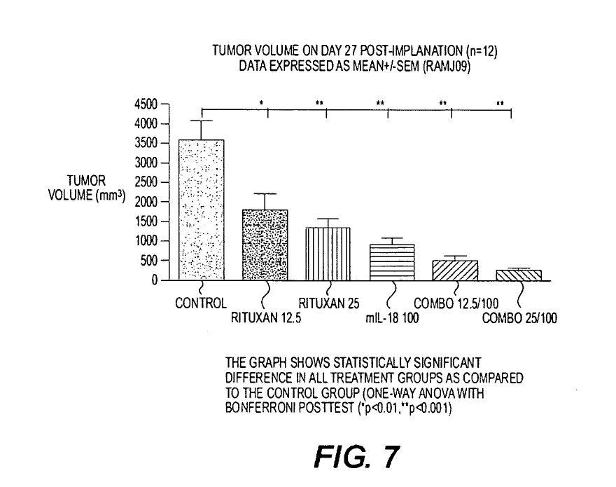

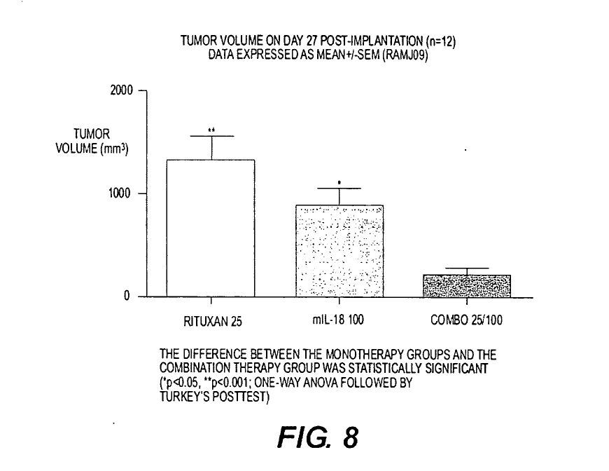

In the second experiment, increasing the number of animals from n=6 to n=12 increased power to determine statistical significance of the additive/synergistic anti-tumor activity in response to combination therapy. The graphs in FIGS. 6A and 6B represent median and mean tumor growth volume. FIGS. 7 and 8 depict statistical analysis of tumor volumes on day 27 post-tumor implantation. The data demonstrate a statistically significant decrease of tumor volume in mice treated with combinational therapy (25/100 μg/mouse), as compared to the Rituxan alone (25 μg/mouse) or mIL-18 (SEQ ID NO:17) monotherapy alone (100 μg/mouse).

This pre-clinical data demonstrates that the combination of IL-18 and rituximab results in synergistic anti-tumor activity. Rituximab was active as monotherapy at the highest dose tested. However, similar levels of activity could be seen when lower doses of rituximab were used in combination with murine IL-18, indicating that the model was sensitive to rituximab and that the response could be enhanced by IL-18. Murine IL-18 enhanced the activity of rituximab, presumably by augmenting ADCC activity in NK cells. Since SCID mice lack both B and T-cell responses, IL-18 is augmenting anti-tumor responses through NK cell activation.

### Example 2

### Combination Therapy of IL-18 with Ofatumumab in Human Lymphoma Xenograft Model

Our goal was to determine if treatment of subcutaneous human Ramos lymphoma (xenograft in SCID mice) with combination therapy of IL-18 (murine) and ofatumumab will result in synergistic anti-tumor activity.

### Background and Methods

Dose-response to ofatumumab mAb was tested in the established Ramos human lymphoma xenograft model (also known as “solid tumor” model, or “subcutaneous tumor” model).
SCID (ICR background, Taconic) female mice received Ramos lymphoma homogenate (0.5 ml of 1:8 homogenate from donor SCID female mice) subcutaneously on day 0. The mice were observed and tumor volumes were measured using calipers twice a week. Tumor volumes were determined using the following formula: (0.5×L)×W² (length of tumor=L, width of tumor=W).
Mice were randomized into therapeutic groups when most tumors reached volume ˜100-150 mm³ on day 17 after implantation (tumors of larger/smaller volumes were excluded from the study). The ofatumumab therapy was administrated intravenously twice a week, and the IL-18 cytokine therapy was administered subcutaneously once a day. The therapeutic groups are listed in Table 1 (below). We have used 6 mice per group in this study.
The read-out from the model is tumor volume measurement and % of cured/regressed/not-cured tumors.
Cured mice are defined as the mice that had tumor volume <10 mm³ for three consecutive measurements. The mice with partial regression are defined as those that had three consecutive measurements showing tumor volume <50% of initial volume. Uncured mice are defined as the mice that do not show improvement in tumor volume (as described above).

TABLE 1
Treatment schedule in the RAMJ10 study.

| | | IL-18 s.c. daily | ofatumumab i.v. |
|---|---|---|---|
| GROUP | # of mice | (ug) | twice a week (ug) |
| 1 | 6 | 0 | 5 |
| 2 | 6 | 0 | 25 |
| 3 | 6 | 0 | 50 |
| 4 | 6 | 100 | 5 |
| 5 | 6 | 100 | 25 |
| 6 | 6 | 100 | 50 |
| 7 | 6 | 100 | 0 |
| 8 (vehicle) | 6 | 0 | 0 |

FIG. 9 shows the effect of ofatumumab (Humax-CD20®) as a monotherapy, or in combination with IL-18 on the growth of subcutaneous Ramos human lymphoma in SCID mice (n=6 mice/group; mean & SD). In addition, data were also expressed as % cured, % regressed and % not cured mice. The definitions are described in methods (above).

The data in FIG. 9 cannot be statistically analyzed because the overall analysis across all time-points using 2-way ANOVA requires all data points for all groups in all time points, which we cannot collect. This is because of the nature of the tumor study—we loose mice (and therefore data points) due to euthanization (tumor volume reached moribund criteria) or due to spontaneous death. Therefore, tumor volume data were statistically analyzed at a selected time-point: on day 28 of the study. This time-point was selected as the latest point of the study where tumor volume data were available from all treatment groups (vehicle group was euthanized due to ethical reasons (extreme tumor volume) at this time).

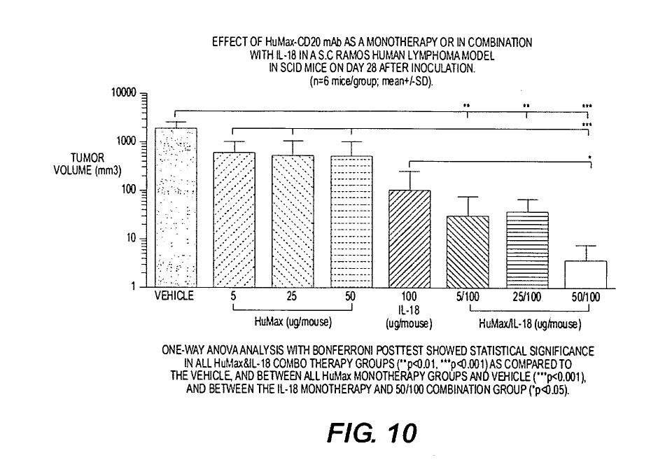

FIG. 10 shows the effect of ofatumumab as monotherapy or in combination with IL-18 in a s.c. Ramos human lymphoma model in SCID mice on day 28 after inoculation. (n=6 mice/group; mean +/−SD). The log transformed data passed all criteria for parametric test processing.

### Conclusions (Parametric Analysis of Log Transformed Data)

Our data demonstrate that monotherapy with ofatumumab monoclonal antibody (mAb) administered at low dose results in a notable inhibitory effect on Ramos lymphoma growth, however this biological effect is not statistically significant. The ofatumumab mAb's effect is, however, significantly potentiated when combined with cytokine IL-18 therapy. The combination therapy with both ofatumumab mAb and IL-18 results in significant tumor growth delay in all dosing groups with the largest significance (p<0.001) in the group with highest dose (50 ug/m ofatumumab+100 ug/m IL-18). The highest combo group (50/100) also shows significantly better outcome than all (low dose) ofatumumab monotherapy groups, and the IL-18 monotherapy group.

In summary, ofatumumab showed synergy with IL-18 combination therapy in Ramos human xenograft model. Data showed dose-dependent anti-tumor activity, similar profile as treatment of same tumor xenograft with combination of IL-18 and rituximab.

### Example 3

### Protocol for Phase I Clinical Trial of IL-18 Combination with Rituximab

Phase I is open-label, dose-escalation study of human IL-18 in combination with standard rituximab therapy investigating the safety and tolerability of 12 weekly ascending doses (1 to 100 μg/kg) of human IL-18 in subjects with CD20+B cell NHL.

Dosing of rituximab and human IL-18 is staggered. Therefore, subjects receive weekly IV infusions of rituximab (375 mg/m²) on Day 1 of Weeks 1 to 4. Human IL-18 is administered as weekly IV infusions on Day 2 of Weeks 1 to 4 and on Day 2 (+/−1 day) of Weeks 5 to 12. The starting dose of human IL-18 is 1 μg/kg, and dose escalation is planned to proceed to a nominal maximum dose of 100 μg/kg.

Dosing within each cohort is staggered with one subject receiving the first dose of rituximab on Day 1 and human IL-18 on Day 2 and then monitored in-house for at least 24 hrs. If there are no safety or tolerability concerns, the next subjects within the cohort is dosed at least 24 hrs later and will also be monitored in-house for 24 hrs after their first human IL-18 dose. On subsequent weeks (Weeks 2 to 12), subjects is monitored for 6 hrs after the human IL-18 dose and then may be released from the clinic. All subjects is dosed at least 2 hrs apart. No more than two subjects per day may be dosed in any cohort.

Three subjects are treated at the first dose level (1 μg/kg/week). If there is no evidence of toxicity greater than Grade 2 with “suspected” or “probable” relationship to study drug after completion of dosing in the cohort (i.e., all three subjects have completed Weeks 1 to 6 of study), three subjects are treated in each subsequent cohort at the following dose levels: 3 μg/kg/week, 10 μg/kg/week, 20 μg/kg/week, 30 μg/kg/week, and 100 μg/kg/week.

For all infusions of rituximab, the complete delivery of the dose, from the initiation of infusion to the end of infusion, must not be less than 4 hrs. Human IL-18 infusion takes place over a two-hour period.

The goal of this study is to determine the maximal biologically effective dose of human IL-18 that is safe when used in combination with standard rituximab treatment in subjects with CD20+ B cell lymphoma. In order to evaluate the dose-response relationship for human IL-18, which was found to be bell-shaped in previous Phase I studies, a dose range of 1 to 100 μg/kg will be used to examine the lower (low dose) and upper end (mid-range or high dose) of the biologically active range in subjects with CD20+ B cell lymphoma.

The dose of rituximab is the standard regimen recommended in the approved labelling for patients with CD20+ B cell NHL. Doses of human IL-18 are selected based on previous Phase I safety, pharmacokinetic, and pharmacodynamic data from studies involving patients with renal cell carcinoma and metastatic melanoma. Robertson, et al., Proc. Am. Soc. Clin. Oncol. 22:178 (abstract 713) (2003); Robertson, et al., J. Clin. Oncol. 22:176s (abstract 2553) (2004); Robertson, et al., J. Clin. Oncol. 23:169s (abstract 2513) (2005); Koch, et al., J. Clin. Oncol. 23:174s (abstract 2535) (2005); Koch, et al., Eur. J. Cancer 4(12):86 (270) (2006). The highest dose tested, 2000 μg/kg administered weekly for up to 24 weeks, produced no significant toxicity such that a maximum tolerated dose was not identified; therefore, pharmacodynamic data are used to select the upper limit of the dose range for this study.

As can be seen from this example, another embodiment of administering IL-18 with an anti-CD20 antibody is a staggered administration, whereby IL-18 and anti-CD20 antibody is given on alternating basis. For avoidance of doubt, either IL-18 or an anti-CD20 antibody may be administered first for in a staggered administration.

| | | |
|---|---|---|
| SEQ ID NO:1 | 2F2 V_{H} | EVQLVESGGGLVQPGRSLRLSCA |
| | | ASGFTFNDYAMHWVRQAPGKGLE |
| | | WVSTISWNSGSIGYADSVKGRFT |
| | | ISRDNAKKSLYLQMNSLRAEDTA |
| | | LYYCAKDIQYGNYYYGMDVWGQG |
| | | TTVTVSS |
| SEQ ID NO:2 | 2F2 V_{L} | EIVLTQSPATLSLSPGERATLSC |
| | | RASQSVSSYLAWYQQKPGQAPRL |
| | | LIYDASNRATGIPARFSGSGSGT |
| | | DFTLTISSLEPEDFAVYYCQQRS |
| | | NWPITFGQGTRLEIK |
| SEQ ID NO:3 | 2F2 V_{H} CDR1 | DYAMH |
| SEQ ID NO:4 | 2F2 V_{H} CDR2 | TISWNSGSIGYADSVKG |
| SEQ ID NO:5 | 2F2 V_{H} CDR3 | DIQYGNYYYGMDV |
| SEQ ID NO:6 | 2F2 V_{L} CDR1 | RASQSVSSYLA |
| SEQ ID NO:7 | 2F2 V_{L} CDR2 | DASNRAT |
| SEQ ID NO:8 | 2F2 V_{L} CDR3 | QQRSNWPIT |
| SEQ ID NO:9 | 11B8 V_{H} CDR3 | DYYGAGSFYDGLYGMDV |
| SEQ ID NO:10 | 2F2 V_{H} CDR1- | DYAMHWVRQAPGKGLEWVSTISW |
| | CDR3 | NSGSIGYADSVKGRFTISRDNAK |
| | | KSLYLQMNSLRAEDTALYYCAKD |
| | | IQYGNYYYGMDV |
| SEQ ID NO:11 | 2C6 V_{H} CDR3 | DNQYGSGSTYGLGV |
| SEQ ID NO:12 | Human V_{H} DP- | EVQLVQSGGGLVHPGGSLRLSCA |
| | 44/D3-10/JH6b | GSGFTFSSYAMHWVRQAPGKGLE |
| | germline | WVSAIGTGGGTYYADSVKGRFTI |
| | sequence | SRDNAKNSLYLQMNSLRAEDMAV |
| | | YYCARDYYGSGSYYYYYYGMDVW |
| | | GQGTTVTVSS |
| SEQ ID NO:13 | Human V_{L} | EIVLTQSPATLSLSPGERATLSC |
| | L6/JK4 | RASQSVSSYLAWYQQKPGQAPRL |
| | germline | LIYDASNRATGIPARFSGSGSGT |
| | sequence | DFTLTISSLEPEDFAVYYCQQRS |
| | | NWPLTFGGGTKVEIK |
| SEQ ID NO:14 | Human V_{H} 3- | EVQLVESGGGLVQPGRSLRLSCA |
| | 09/D4-11/JH6b | ASGFTFDDYAMHWVRQAPGKGLE |
| | germline | WVSGISWNSGSIGYADSVKGRFT |
| | sequence | ISRDNAKNSLYLQMNSLRAEDTA |
| | | LYYCAKDIDYYYYYYGMDVWGQG |
| | | TTVTVSS |
| SEQ ID NO:15 | Human V_{L} | EIVLTQSPATLSLSPGERATLSC |
| | L6/JK5 | RASQSVSSYLAWYQQKPGQAPRL |
| | germline | LIYDASNRATGIPARFSGSGSGT |
| | sequence | DFTLTISSLEPEDFAVYYCQQRS |
| | | NWPITFGQGTRLEIK |

1-9. (canceled)

10. A method of treating or preventing cancer in a patient, comprising the step of: administering to the patient: (i) a human IL-18 polypeptide (SEQ ID NO:16); and (ii) rituximab.

11. The method as claimed in claim 10 wherein the administration of the human IL-18 polypeptide (SEQ ID NO:16) and rituximab is simultaneous.

12. The method as claimed in claim 10 wherein the administration of the human IL-18 polypeptide (SEQ ID NO:16) and rituximab is sequential, wherein the human IL-18 polypeptide (SEQ ID NO:16) is administered first.

13. The method as claimed in claim 10 wherein the administration of the human IL-18 polypeptide (SEQ ID NO:16) and rituximab is sequential, wherein the antibody is administered first.

14. The method as claimed in claim 10 wherein the administration of the human IL-18 polypeptide (SEQ ID NO:16) and the antibody is staggered.

15. The method as claimed in claim 10 wherein the cancer is a B cell lymphoma.

16. A method of claim 10 wherein the cancer is selected from the group consisting of NHL (non-Hodgkin's lymphoma), B cell lymphoblastic leukemia/lymphoma, mature B cell neoplasms, B cell chronic lymhocytic leukemia (CLL)/small lymphocytic lymphoma (SLL), B cell prolymphocytic leukemia, lymphoplasmacytic lymphoma, mantle cell lymphoma (MCL), follicular lymphoma (FL), including low-grade, intermediate-grade and high-grade FL, cutaneous follicle center lymphoma, marginal zone B cell lymphoma (MALT type, nodal and splenic type), hairy cell leukemia, diffuse large B cell lymphoma, Burkitt's lymphoma, plasmacytoma, plasma cell myeloma, post-transplant lymphoproliferative disorder, Waldenstrom's macroglobulinemia, anaplastic large-cell lymphoma (ALCL), T-cell Non-Hodgkin's lymphoma; and melanoma.
